(19) United States
(12) Patent Application Publication
(10) Pub. No.: US 2002/0127608 A1
(43) Pub. Date: Sep. 12, 2002

(54) Method for selecting compounds from a combinatorial or other chemistry library for efficient synthesis

(76) Inventors: Mario G. Cardozo, Brookfield, CT (US); Edward T. Graham, New Haven, CT (US); Stephen P. Jacober, Danbury, CT (US)

Correspondence Address: BOEHRINGER INGELHEIM CORPORATION, 900 RIDGEBURY ROAD, P O BOX 368, RIDGEFIELD, CT, 06877, US

(21) Appl. No.: 10/043,707

(22) Filed: Jan. 10, 2002

Related U.S. Application Data
(63) Continuation of application No. 09/502,921, filed on Feb. 11, 2000, now abandoned.

(51) Int. Cl. G01N 33/53; G06G 7/48; G06G 7/58

(52) U.S. Cl. 435/7.1; 703/12; 702/22

(57) ABSTRACT

A method for determining the most efficient way to synthesize compounds that form a combinatorial, chemical library is disclosed and described, with an example of the use of such method to develop a synthesis scheme.

### BACKGROUND OF THE INVENTION

The present invention provides a method for selecting compounds (a subset) out of a large group or number of compounds (the original set). The selection or subset will be smaller than the initial set of compounds and will be selected so that the new subset can be efficiently synthesized as a group. In particular, the method of the present invention can be used to create subsets from combinatorial chemical libraries that can be synthesized together. The selection of compounds using the method of the present invention will not only produce a group of compounds in a manageable number, but will also produce a group of compounds that can be efficiently synthesized, i.e., use common reagents.

It is now possible with the developing technologies of combinatorial chemistry to generate potentially enormous chemical libraries of structurally diverse molecules. Combinatorial chemistry assembles selected sets of reagents in combinatorial arrangements, using appropriate chemical reactions, into a diverse library of related compounds. However, because of the size of these libraries, it is not commercially feasible to synthesize all of the potential molecules and test them for biological activity. Therefore, anyone attempting to test such libraries for biological activity is faced with the problem of devising a method to perform a selection of a subset of compounds from a large combinatorial library, such that the subset posesses maximum chemical diversity (in order to obtain meaningful data or structure activity relationships) while maintaining practical size limitations. Additionally, it is also desirable to obtain a subset that can be synthesized efficiently, that is, a subset that can be synthesized using a minimum number of reagents. A method which could produce a subset library containing an efficient chemically synthesizable set of diverse molecules amenable to automated combinatorial chemistry would be an improvement over methods which are currently available.

Previous subset-selection methods have all been based on algorithms measuring library similarity/dissimilarity. (Valerie J. Gillet, David J. Wild, Peter Willett, and John Bradshaw, “Similarity and Dissimilarity Methods for Processing Chemical Structure Databases”, The Computer Journal, 1998, Vol. 41, No. 8, 547-558; Andrew C. Good and Richard A. Lewis, “New Methodology for Profiling Combinatorial Libraries and Screening Sets: Cleaning Up the Design Process with HARPick”, J. Med. Chem., 1997, 40, 3926-3936; Richard E. Higgs, Kerry G. Bemis, Ian A. Watson, and James H. Wikel, “Experimental Designs for Selecting Molecules from Large Chemical Databases”, J. Chem. Inf. Comput. Sci., 1997, 37, 861-870; Wendy A. Warr, “Combinatorial Chemistry and Molecular Diversity. An Overview”, J. Chem. Inf Comput. Sci., 1997, 37, 134-140; Young, S. S., Sheffield, C. F., Farmen, M., “Optimum Utilization of a Compound or Chemical Library for Drug Discovery”, J. Chem. Inf. Comput. Sci., 1997, 37, 892-899; Holliday, J. D., Ranade, S. S., Willett, P., “A fast Algorithm for Selecting Sets of Dissimilar Molecules from Large Chemical Databases”, Quant. Struc.-Act. Relat., 1996, 14, 501-506; Martin, E. J., Barney, J. M., Siani, M. A., Spellmeyer, D. C., Wong, A. K., Moos, W. H., “Measuring Diversity: Experimental Design of Combinatorial Libraries for Drug Discovery”, J. Med. Chem., 1995, 38, 1431-1436.) Although these methods claim to produce maximally diverse subsets, no attempt has been made to take into consideration the practicality of automated chemical synthesis. As a result, the subset produced still may not be useful even though it may be considerably smaller than the original library because of the inefficiencies involved in synthesizing all of the molecules selected; that is, too many reagents are required to prepare the subset of the chemical library. The excess in the number of reagents results from the fact that potentially many of these reagents have to be used for only a few, specific combinations, or, in some cases, only one time. This situation increases the manipulation of reagents, as well as requires complex and less practical robotic operations. Finally, some of these methods (genetic algorithms, neural networks, random sampling) do not produce unique solutions.

### BRIEF SUMMARY OF THE INVENTION

The method presented here is based on reagent frequency analysis and can be applied to any library of molecules distributed in any given diversity space (cluster, cell-based, or any other distribution). Compound selection by reagent frequency distribution can produce a unique, maximally diverse set of molecules that adequately represents the library while requiring the least amount of compounds to be synthesized. Minimum compound generation results in a savings in both time of synthesis and cost of materials. This invention always results in a discrete solution, which can be used for any given library size as well as any combination of reagents. This invention is also readily adaptable to robotic automation.

### BRIEF DESCRIPTION OF THE DRAWINGS

FIG. 1 is a schematic drawing demonstrating that the cross-product of two independently derived lists do not necessarily cover all clusters.

FIG. 2 is a schematic drawing showing groupings of possible compounds.

FIG. 3 is a density map.

FIG. 4A is a density map.

FIG. 4B is a density map.

FIG. 5A is a plate density map.

FIG. 5B is a table.

FIG. 6A is a plate density map.

FIG. 6B is a table.

FIG. 7A is a plate density map.

FIG. 7B is a table.

FIG. 8A is a plate density map.

FIG. 8B is a table.

FIG. 9A is a plate density map.

FIG. 9B is a table.

FIG. 10A is a plate map.

FIG. 10B is a plate map.

FIG. 11A is a plate map.

FIG. 11B is a plate map.

FIG. 12A is a plate map.

FIG. 12B is a plate map.

### DETAILED DESCRIPTION OF THE INVENTION

A chemical library is partitioned into regions of “similarity” based on whole molecule diversity analysis (by any method and to whatever degree is desired). For example molecular weight may be used (all compounds having a similar molecular weight), chemical structure (classifications) or any other criterion or group of criteria that provides a rational basis for grouping or placing compounds into discrete sets or clusters. Substituent frequency analysis of molecular fragments (reagents) used to prepare the whole molecule is applied to this partitioned chemical library resulting in ordered (by decreasing frequency) lists (one list for each substituent location) of fragments based on their frequency across the clusters. These ordered lists are then further organized to minimize resources required for synthesis (e.g., number of reagents and/or number of reaction plates). When this approach is used to guide reagent selection for the synthesis of a chemical library, the subset generated by these lists will produce the minimum number of combinations for a corresponding degree of molecular diversity using a minimum number of resources.

After a chemical library is partitioned into regions of similarity, i.e., “clusters”, the frequency distribution procedure of the present invention is described with reference to the following structure.

Consider the following structure (Formula A) as an example of a parent compound with three substituent group locations: R1, R2 and R3.

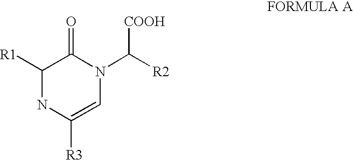

R1, R2 and R3 represent locations for substitution on the structure designated Formula A.

The first step is to generate a rank ordered (high to low) frequency distribution list of substituents for each substituent group location (i.e., R1, R2 and R3). This is accomplished by performing a series of steps. (Substituent and reagent are used interchangeably in this scheme.)

### STEP A

The first step is to determine the most frequent substituent within a specific substituent group location (e.g., R1) across the clusters. That is, what is the specific substituent that occurs in the most number of clusters. That substituent is considered to provide the most chemical diversity for that location when combined with the other substituents at locations R2 and R3 because it is represented in more different clusters than any other substituent for that group location.

### STEP B

The second step is to eliminate all of the clusters and their contents (all other compounds in those clusters) for that substituent determined in step A. The rationale behind such elimination is that all compounds within any given cluster are assumed to be equivalent for the chemical space used to perform the partition (to make up a cluster). Therefore, it is most efficient to have only one compound from each cluster, rather than several compounds from the same cluster.

### STEP C

At this point, Steps A and B are repeated to determine the second most frequent, the third most frequent, etc. This produces the first ordered list.

### STEP D

This entire procedure (steps A through C) is repeated for each group substituent location (to generate subsequent or, for convenience, 2^{nd}, 3^{rd}, . . . lists).

### STEP E

The cross-product of the lists is generated and the clusters covered by the compounds formed are removed from the library. Then, next generate subsequent or 2^{nd}, 3^{rd} and . . . lists.

### STEP F

Repeat Steps A through F until all clusters are covered.

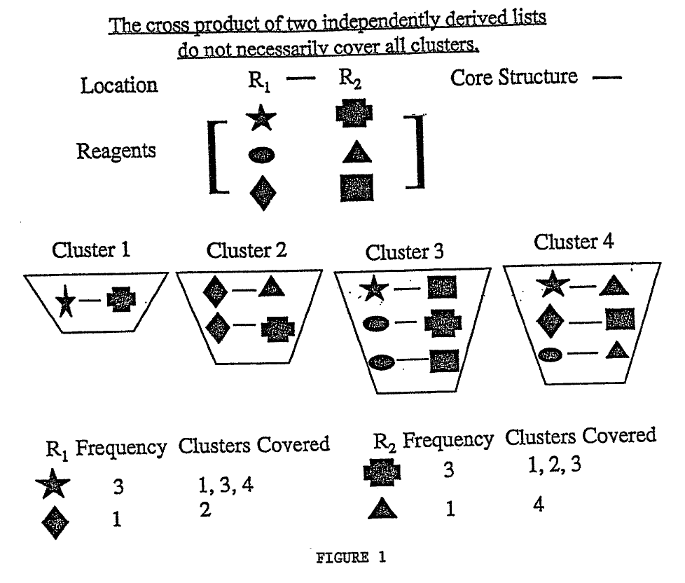

Referring to FIG. 1, R1 consists of the following reagents: “star”, “oval” and “diamond.” R2 consists of the following reagents: “cross”, “triangle” and “square.” There are four clusters shown, cluster 1 having one member, cluster 2 having two members, cluster 3 having three members and cluster 4 having three members. Taking R1 and using STEP A, the most frequent substituent for R1 is star. Looking at the clusters, R1 is star in clusters 1, 3 and 4. Using STEP B, those clusters are eliminated. STEP A is then used to determine the second most frequent substituent. Since only cluster 2 remains, the second most frequent substituent for R1 is diamond. Then, using STEP B, cluster 2 is eliminated, and since all clusters have been eliminated for R1, no further action is necessary. The ranking for R1 is star first (present in three (3) clusters) and diamond second (present in one cluster). STEPS A, B and C are then performed for R2.

The results, also shown in FIG. 1, are that when R2 is cross, this substituent is found in three clusters (cluster 1, cluster 2 and cluster 3), and those clusters are eliminated. In the remaining cluster, cluster 4, the most frequent reagent for R2 is triangle. All clusters have now been eliminated and the ranking for R2 is cross first and triangle second.

The resultant frequency lists can have any number of elements from 1 to m, as shown below in Table I [the total number of “r”s (e.g. in R1) groups depending on the diversity of the substructures (reactants)]. Note that when “R” is implied to contain elements (r), it will be denoted as a vector R. As noted before, R1 represents a specific location on the parent molecule.

TABLE I

| | | frequency (# of clusters |
|---|---|---|
| | Rank of Substituents | containing substituent) |
| most frequent substituent | R_{1,1} | f₁ |
| second most frequent | R_{1,2} | f₂ |
| third most frequent | R_{1,3} | f₃ |
| m′ost frequent | R_{1,m} | fm |
| | | N |

### EXAMPLE A

Let R1 have n1 elements and R2 have n2 elements. Then the “R” group frequency lists created are crossed (e.g. R1×R2) to form a library with (n1×n2) number of compounds. The number of partitions (clusters, cells, etc.) covered by these compounds are determined based on the original distribution of the entire compound library within the partitions. Because the “R” groups frequency is determined independently of each other, it would be unlikely that all of the clusters would be represented. This is demonstrated schematically by reference to FIG. 1.

FIG. 1 shows schematically the nine possible combinations of crossing R1 with R2, e.g., star crossed with triangle. The nine combinations are then partitioned into four clusters, shown as Cluster 1, Cluster 2, Cluster 3, and Cluster 4. Based on reagent frequency analysis, note that the star and after the star the diamond are sufficient to cover all of the clusters for the R1 location. That is, three clusters have R1 as star, and one cluster has R1 as diamond, and all four clusters have at least one member with R1 as a star or as a diamond. The cross and after the cross the triangle are sufficient to cover all of the clusters for the R2 location. However, even though the cross product based on reagent frequency analysis results in four elements, these do not produce sufficient combinations to cover all of the clusters (cluster 3 with three (3) elements is not covered).

Accordingly, a new dataset is created whose contents are the clusters not covered by the compounds produced by the crossed frequency list (in the above example and as shown in FIG. 1, cluster 3). Note, this dataset is created by eliminating all of the clusters covered and, as well, as all of the remaining compounds with reagents identified in the frequency lists.

Steps A and B from above are then repeated for the new dataset to create a secondary frequency list.

Then, the above process is continually repeated (to generate tertiary, quaternary, . . . etc. for m lists) until one of the following criteria are met:

a) all of the clusters are covered (resulting in p substituents); [It may not be possible to generate sufficient lists that result in all clusters being covered. Because R group substituents are not deleted from clusters remaining after the list created by the cross product is generated, the same frequency lists can be continually repeated because no new combinations are being generated. Alternative methods can be used to generate lists that cover all clusters. These lists may not produce an efficient robotic solution for synthesis (as determined by number of plates to cover clusters, method description below)]

b) the frequency counts of the R groups are low (e.g., the most frequent R group has a count of 2 or 3); or

c) the total number of elements in the frequency list numbers whatever number of r substructures (reagent groups) that may be desired or p (an arbitrary number which can be adjusted to account for a sufficiently large enough selection of substructures for library generation).

The “R” substituent groups are ranked from 1 top as determined by the order in the primary, secondary, tertiary, . . . lists.

Suppose, for example, a combinatorial library has 281 substituents (reagents) in R1, 219 in R2, and 150 in R3. Therefore, the total number of compounds possible to make is (281×219×150=)9,230,850. After frequency analysis, R1 has a primary list containing 50 substituents, the secondary list 30 and the tertiary list 16 (50+30+16=96) then the substituents from the primary list will be 1−50, the secondary list 51−80 and the tertiary list 81−96. Let's suppose R2 has 65 substituents in the primary list, 20 in the secondary, and 11 in the tertiary and R3 has just 45 substituents. This could be all of the reagents necessary to cover all of the clusters or the count required to cover some percent of the clusters, or an arbitrary number of reagents.

Crossing the “R” group lists to create a list of compounds to synthesize, i.e.,

{(R 1primary+secondary+. . . )×(R2 primary+secondary +. . . )×(R3 primary+secondary+. . . )×( . . . )}

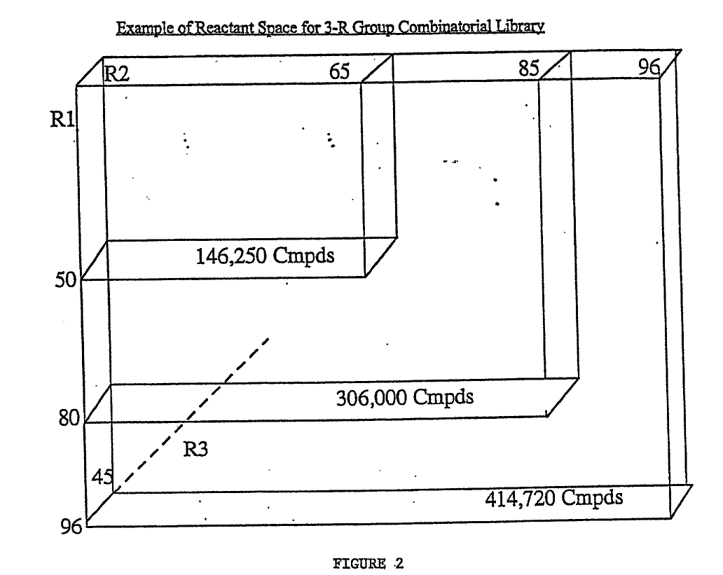

would result in (96×96×45) 414,720 potential compounds to cover all clusters. This is substantially less than the total number of compounds in the virtual library (9,230,850). In the case of less than full coverage, other potential schemes for this example might be (50×65×45) 146,250, or (80×85×45) 306,000. This is shown schematically in FIG. 2.

The list of compounds created by the crossed R group frequency lists is then sorted by Cluster, Rank-R1, Rank-R2, Rank-R3, . . . etc. The first observation in every cluster in the sorted list is selected. This compound will be assumed to be representative of that cluster and has the property of being composed of substituent groups that are represented in more different clusters. The number of compounds required to represent the entire library now equals the number of clusters generated (as determined by the partitioning method e.g. nonparametric: density estimation, Kohonen Neural Network, Genetic Algorithm etc.).

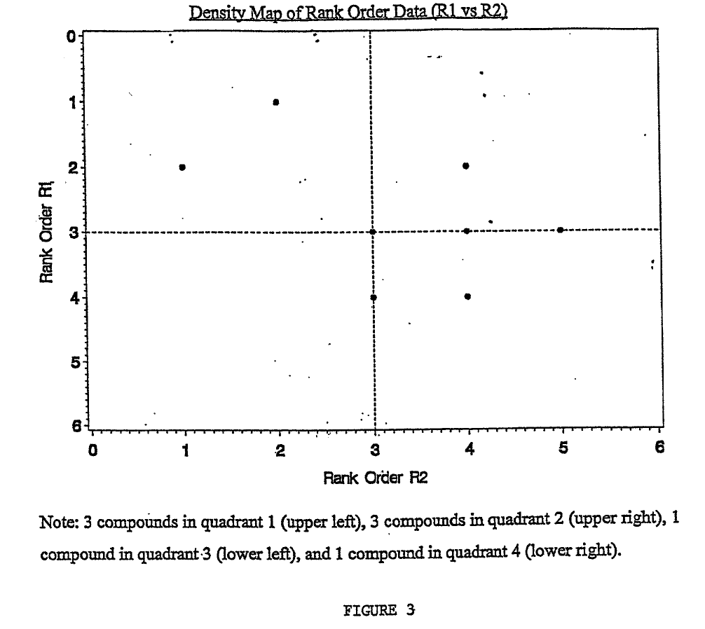

A graph is then created, and the rank of all of the members of each R group is plotted on a separate axis of the above dataset created above. The plot of coordinate ranks on this graph will create a density map. The data set forth in Table II below provided the points that are co-ordinates in FIG. 3. The objective is to maximize the density to the smallest area of the graph. This is accomplished by re-ordering the coordinate ranks such that the re-mapped ranks occupy as small a region as possible on the map. This, therefore, provides the optimum condition for the most efficient synthesis of compounds covering most clusters.

TABLE II
Example: Rank Data for Two R Groups of Reactants (R1 and R2)

| Observation | Frequency Rank Order for | Frequency Rank Order for |
|---|---|---|
| ID | Reactants at R1 | Reactants at R2 |
| 1 | 1 | 2 |
| 2 | 2 | 1 |
| 3 | 2 | 4 |
| 4 | 3 | 3 |
| 5 | 3 | 4 |
| 6 | 3 | 5 |
| 7 | 4 | 3 |
| 8 | 4 | 4 |

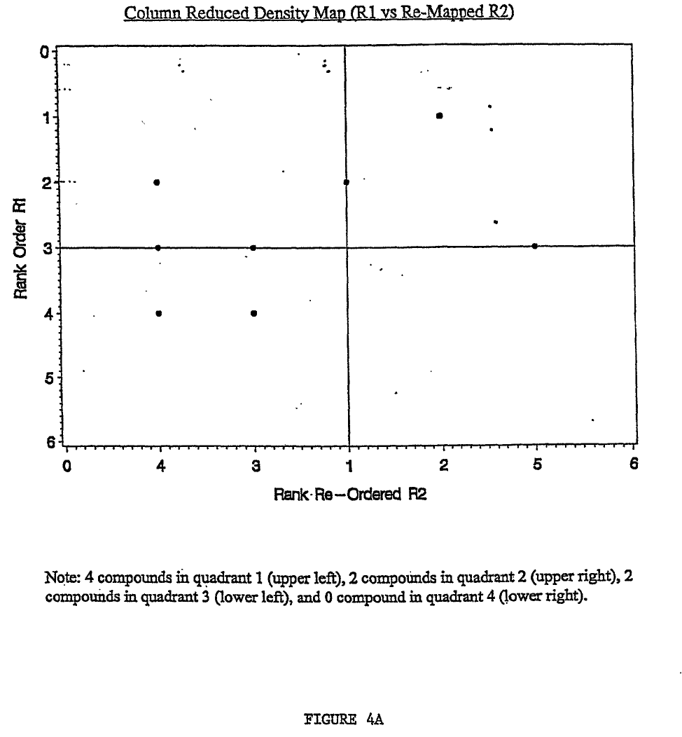

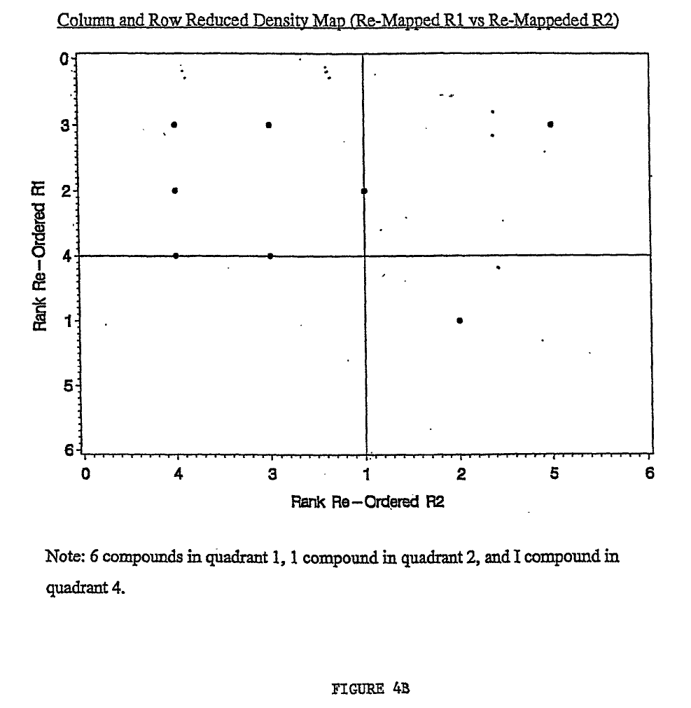

The number of compounds in each row is determined and the ranking from high to low is re-ordered. The re-ordered ranking will result in a new density map, (as shown in FIG. 4A and FIG. 4B.) the re-ordered data is presented in Table III, below. This will increase the density to the maximum allowable for this particular set of selected compounds. However, it is possible that the optimal solution may result by re-ordering the row first, and then the columns. Both scenarios should be checked. The scenario that produces the highest density region using the minimum number of reagents should be selected.

TABLE III
Re-Mapped R Group Data

| Observation | Frequency Re-Rank Order | Frequency Re-Rank Order |
|---|---|---|
| ID | for Reactants at R1 | for Reactants at R2 |
| 1 | 4 | 4 |
| 2 | 2 | 3 |
| 3 | 2 | 1 |
| 4 | 1 | 2 |
| 5 | 1 | 1 |
| 6 | 1 | 5 |
| 7 | 3 | 2 |
| 8 | 3 | 1 |

At this point, the density map should be divided into grids of desired dimensions. For example, the density map can be divided into ninety-six grids {12×8, 8×12, 4×[(6×4) or (4×6)]} and the number of compounds in each grid is counted. The grids are then sorted and the most dense grids are selected. The minimum number of plates required (least amount of work) to cover a specified number of clusters. If plates having more than ninety-six wells are to be utilized, then obvious adjustments to be made.

In a multi-component system with greater than two R group locations, the above procedures will have to be repeated for each pair of possible R group combinations to guarantee the optimum maximum density. The number of possible combinations can be calculated from the following

$$( \begin{matrix}m \\ n\end{matrix} ) = \frac{m !}{n ! ( m - n ) !}$$

where m=the number of R group locations and n=2 (e.g. 2 dimensional 96 well plate). For a 3-R group substituent problem where optimum maximum density of a 96 well plate was required, three density maps would have to be evaluated (R1×R2 by R3, R2×R3 by R1, and R1×R3 by R2).

### Frequency Distribution Method Variations

A parent compound has two substituent positions (R1 and R2). R1 has 281 substituents and R2 has 219 substituents. All together there are 61,539 possible combinations (R1×R2). The 61,539 possible structures are partitioned into 898 regions of similarity (clusters). Three different variations based on the Frequency Distribution Method of substituents for each of the two substituent locations are performed on the 898 clusters. The variations will be noted as Method 1, Method 2, and Method 3.

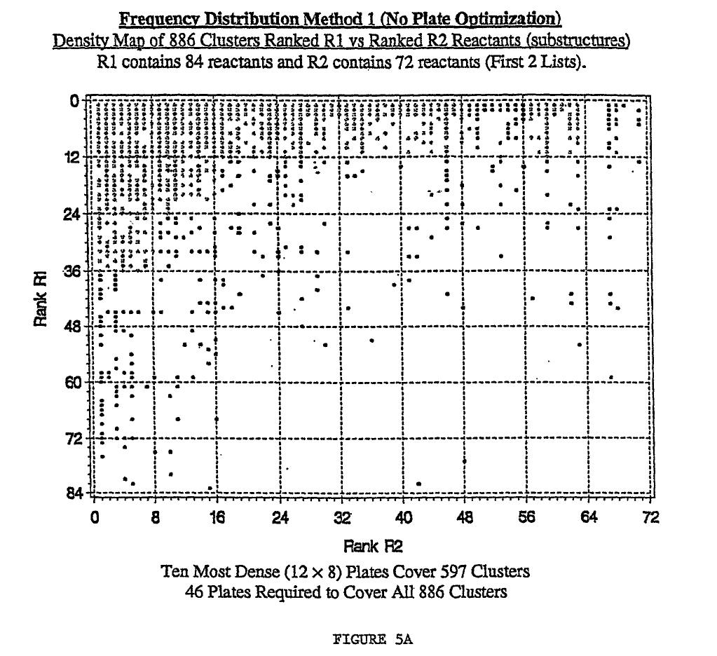

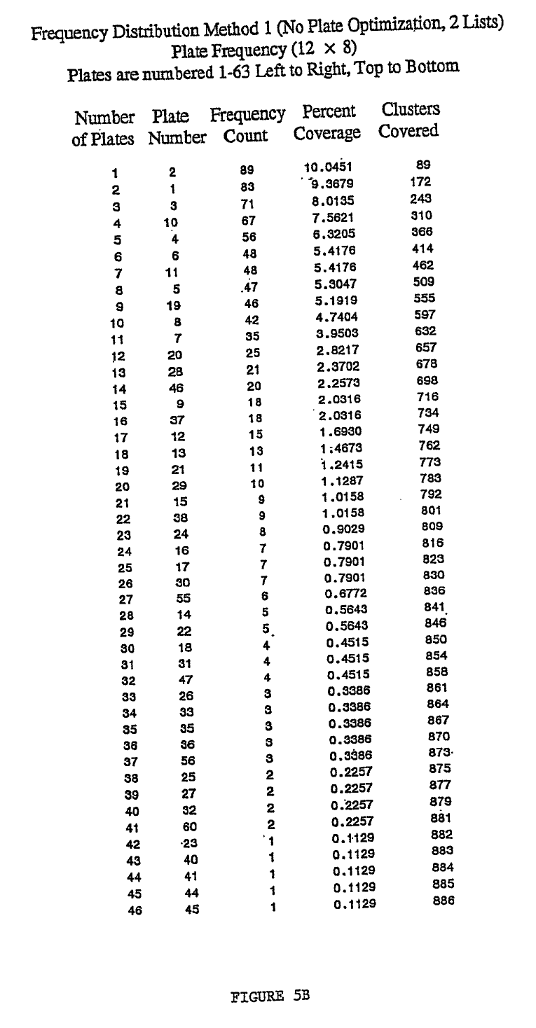

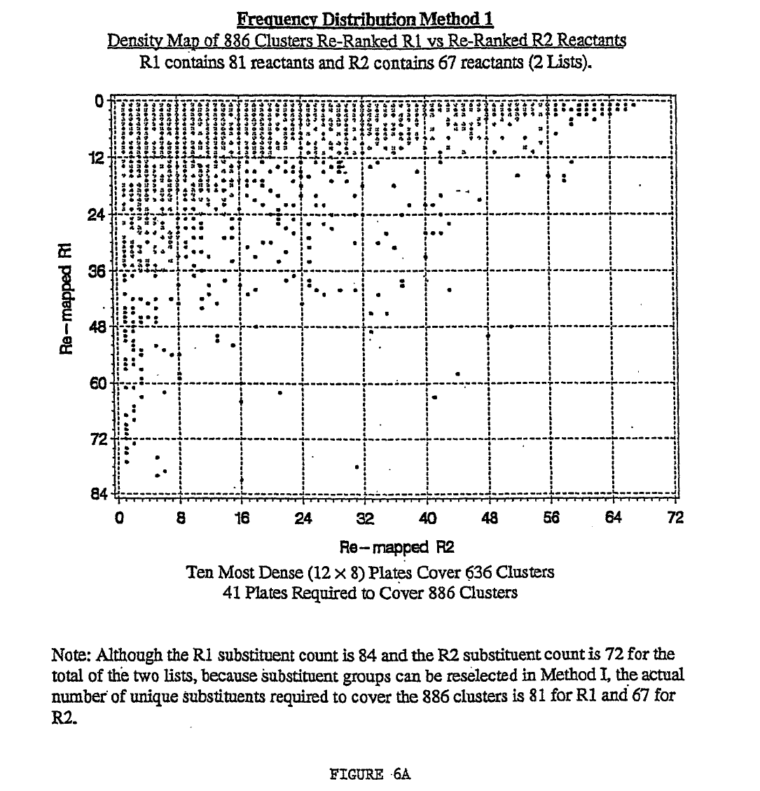

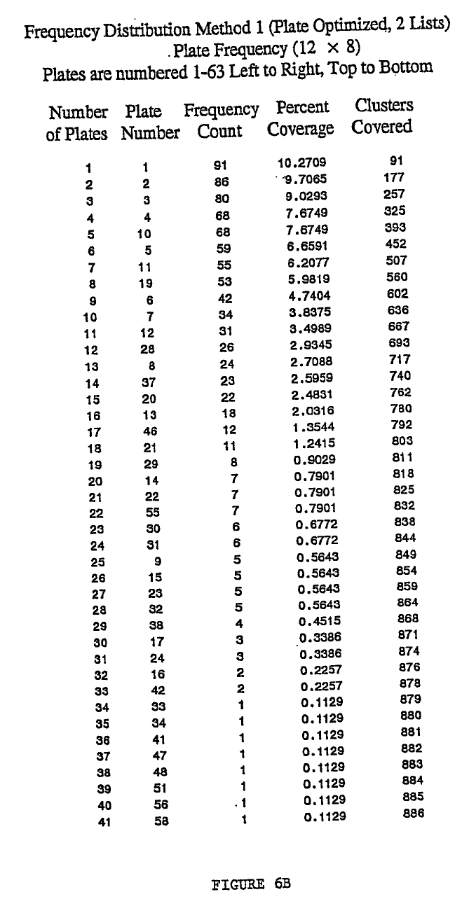

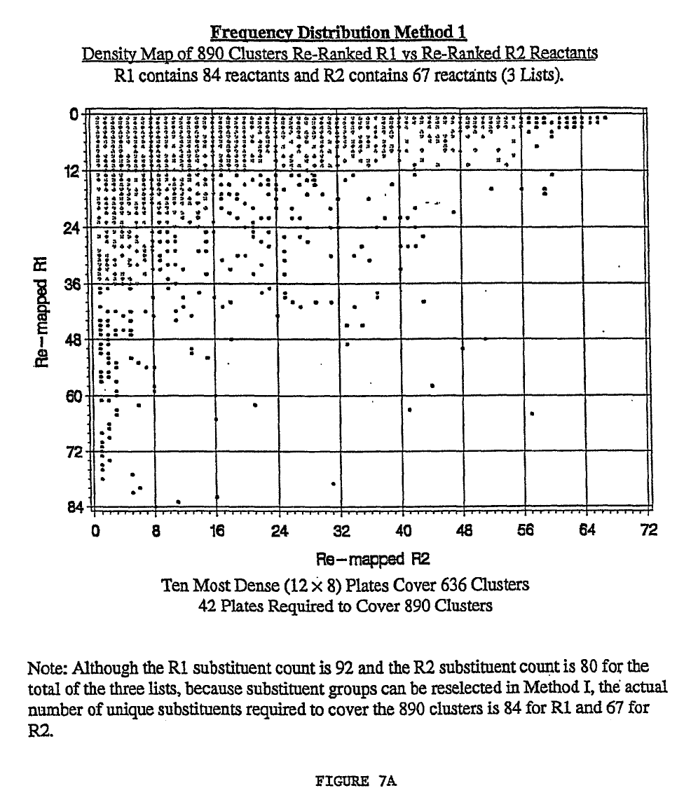

Method 1 is as described above. As mentioned above, no improvement in number of clusters covered results after the tertiary list is generated. Because substituent reagents are not eliminated from the dataset containing the remaining clusters, it is possible to loop through with the same frequency groups being selected again due to the independent nature of the frequency lists. FIG. 5A and FIG. 5B show the results for Method 1. However, density map optimization was not performed. FIG. 6A and FIG. 6B show Method 1 with two list iterations and density map optimization. FIG. 7A and FIG. 7B show Method 1 with three list iterations and density map optimization. Table IV is shown below with a summary of the results using Method 1.

TABLE IV
Method 1

| | R1 | R2 | | Percent of Total | Required |
|---|---|---|---|---|---|
| | # of Reagents | # of Reagents | | Clusters | Cmpds to |
| | per Frequency | per Frequency | Clusters Covered | Covered | Synthesize |
| List | List | List | (Cummulative) | (Cummulative) | (Cummulative) |
| 1 | 73 | 61 | 844 | 93.9% | 4,453 |
| 2 | 11 | 11 | 886 | 98.6% | 6,048 |
| 3 | 8 | 8 | 890 | 99.1% | 7,360 |
| 4 | 6 | 5 | 890 | 99.1% | 8,330 |
| 5 | 6 | 5 | 890 | 99.1% | 8,330* |

*Note:
List 5 is a repeat of List 4 with the same reagents and will continue to repeat ad infinitum.

The improvement in partitions covered with plate number for density map optimization is readily apparent.

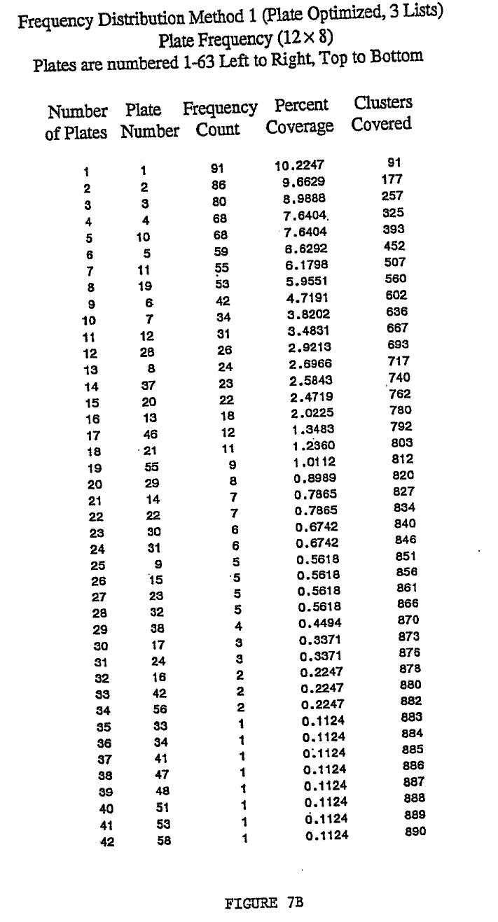

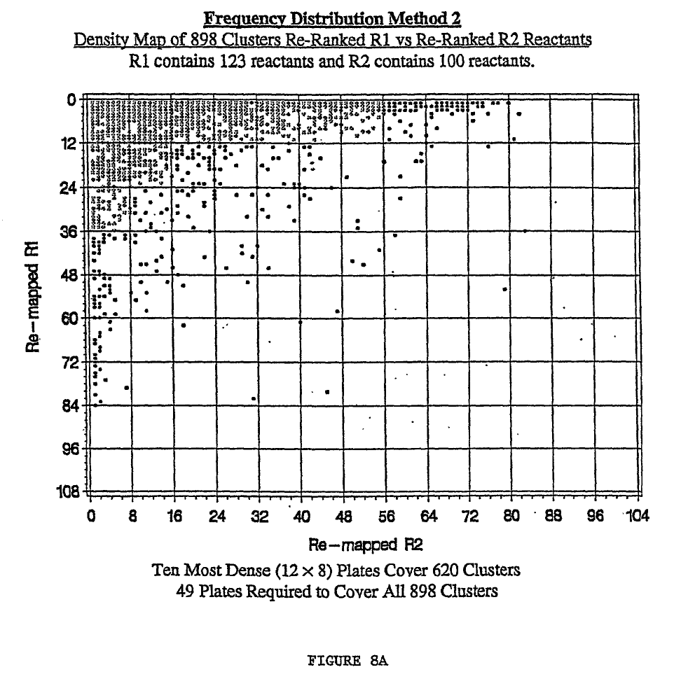

Method 2 is identical to Method 1 except that when the new dataset of clusters not covered by the previous list(s) is created, in addition to eliminating the clusters that are covered, those structures that include substituent reagents already included in the frequency list are also eliminated from the clusters that remain. Accordingly, the entire structure is eliminated, which eliminates other substituent locations from flier frequency analysis also. This will always result in all clusters being covered when all of the reagent lists are crossed for R1 and R2. See FIG. 8A and FIG. 8B, and Table V (below) for the results of Method 2.

TABLE V
Method 2

| | R1 | R2 | | Percent of Total | Required |
|---|---|---|---|---|---|
| | # of Reagents | # of Reagents | | Clusters | Cmpds to |
| | per Frequency | per Frequency | Clusters Covered | Covered | Synthesize |
| List | List | List | (Cummulative) | (Cummulative) | (Cummulative) |
| 1 | 73 | 61 | 844 | 93.9% | 4,453 |
| 2 | 19 | 12 | 890 | 99.1% | 6,716 |
| 3 | 12 | 9 | 893 | 99.4% | 8,528 |
| 4 | 8 | 8 | 896 | 99.7% | 10,080 |
| 5 | 6 | 6 | 896 | 99.7% | 11,328 |
| 6 | 4 | 3 | 898 | 100% | 12,078 |
| 7 | 1 | 1 | 898 | 100% | 12,300 |
| 8 | 0 | 0 | | | |

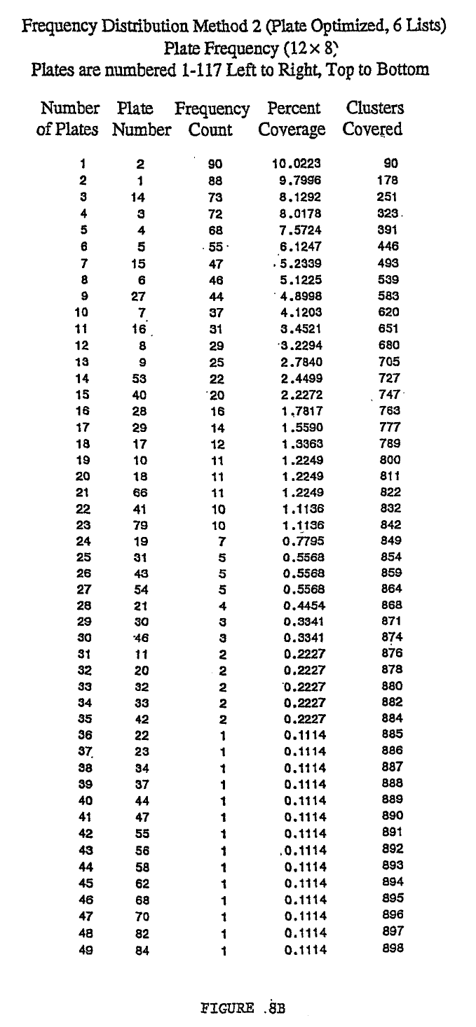

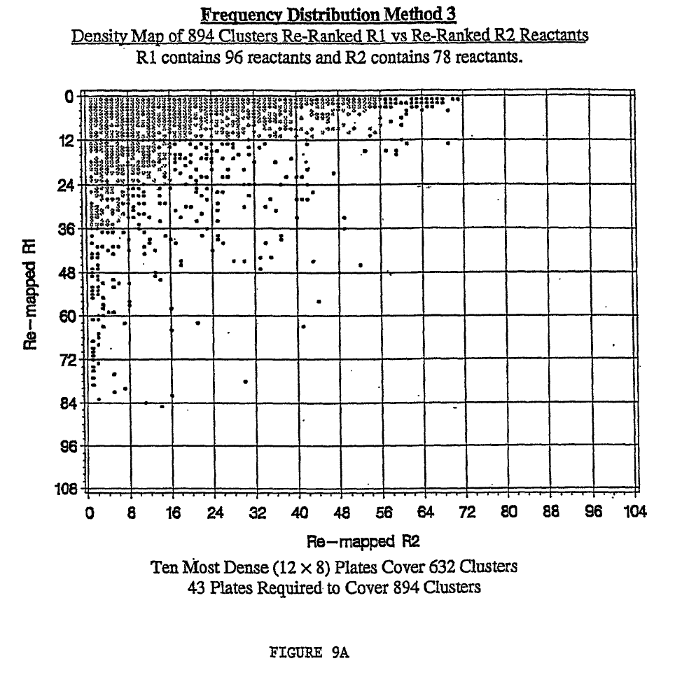

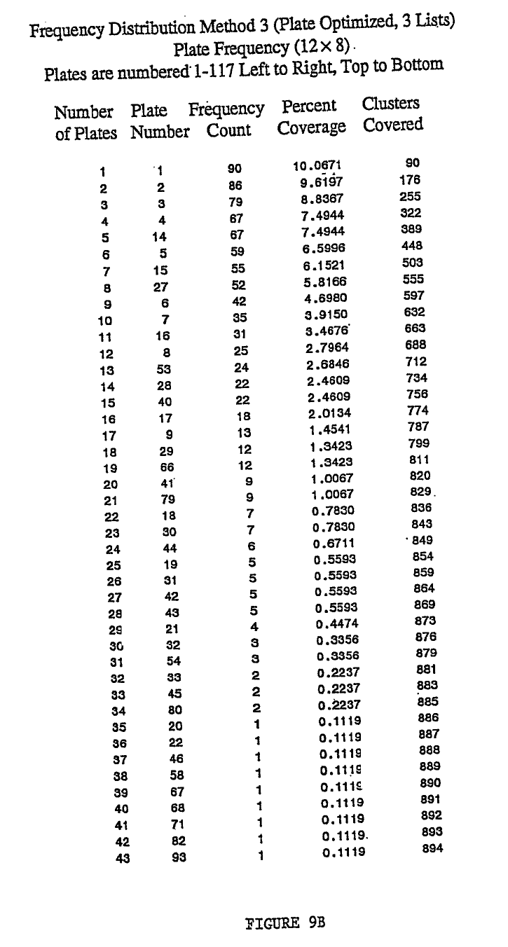

Method 3 is also as outlined above by Method 1, except that when creating the new dataset of clusters not covered by the previous list(s), in addition to eliminating the clusters that are covered, those substituents are eliminated from the frequency lists but only for a particular location (R1 or R2). The entire structure is not eliminated as in Method 2. See FIG. 9A and FIG. 9B and Table VI (below) for results of Method 3.

TABLE VI
Method 3

| | R1 | R2 | | Percent of Total | Required |
|---|---|---|---|---|---|
| | # of Reagents | # of Reagents | | Clusters | Cmpds to |
| | per Frequency | per Frequency | Clusters Covered | Covered | Synthesize |
| List | List | List | (Cummulative) | (Cummulative) | (Cummulative) |
| 1 | 73 | 61 | 844 | 93.9% | 4,453 |
| 2 | 18 | 12 | 891 | 99.2% | 6,643 |
| 3 | 5 | 5 | 894 | 99.5% | 7,488 |
| 4 | 0 | 0 | | | |

When a large collection of compounds are partitioned by any procedure based on some algorithm of similarity, there will likely be a wide distribution in the number of compounds populated per partition (cell, cluster, etc.). Since compounds are distributed according to their similarity, partitions with small populations are the least like other compounds in the collection. For example, the most uncommon compounds will occur alone in separate partitions. These partitions are selected last during the Frequency Distribution Method(s). These compounds are the most expensive to synthesize in terms of unique reagents required and computer time to locate. (These unique reagents will have low frequencies and therefore will be selected in the later lists. Also, they will likely be located on plates which have higher cluster information, i.e., cluster duplication from previous plates.) Note that in all three variations of the frequency distribution method described above, plates populated first cover more partitions (clusters) than plates toward the end. To cover all of the partitions, a high price in terms of compounds required to be synthesized is paid to represent these clusters containing more unique compounds. For example, in Method 1 as shown in FIG. 7B, after 33 plates there are 880 clusters that are represented. To cover the next 10 clusters require 9 more plates (or 864 additional compounds to be synthesized). A determination would need to be made on whether it would be more work for a robot to generate these 864 compounds (and subsequent testing) or have a chemist synthesize 10 compounds to represent these clusters. Also note from FIG. 8B and the summary below (Table VII), that Method 2 also covers 880 clusters after 33 plates and requires an additional 16 plates (or 1,536 compounds) to cover all of the clusters 898 (18 more clusters than 33 plates cover).

TABLE VII
Methods Summary Statistics of Plate Optimized Selections

| | | | | | | Cmpds |
|---|---|---|---|---|---|---|
| | | | | | | Required |
| | | | | No. of | | to be |
| | No. of | No. of | Max- | Plates | No. of | Synthesized |
| | Rea- | Rea- | imum | Required | Frequency | (No. of |
| Me- | gents | gents | Clusters | To Cover | Lists | Plates |
| thod | At R1 | At R2 | Covered | Maximum | Generated | × 96) |
| 1 | 84 | 67 | 890 | 42 | 3 | 4,032 |
| 2 | 123 | 100 | 898 | 49 | 6 | 4,704 |
| 3 | 96 | 78 | 894 | 43 | 3 | 4,128 |

All three variations of frequency distribution method produce similar results. While Method 1 allows reagents to be repeatedly selected as long as it represents the maximum frequency (most common reagent across clusters), Method 2 and Method 3 will select unique reagents as the iteration process continues. Method 2 will always eventually generate lists sufficient to cover all clusters where Method 1 and 3 cannot necessarily be driven to cover all clusters. There are several criteria that should be investigated when determining which method to use:

computation time (the more lists that have to be generated the more time is required);

cluster coverage (only Method 2 guarantees that all clusters will be covered);

cluster coverage by number of plates material;

resources (reagents, plates, etc.); and

number of compounds required to be synthesized (and tested).

In general, although all of the methods give similar results for early iterations, Method 1 will produce lists with the minimum number of reagents required and thus less expensive in terms of computer time, resources, and required number of compounds that must be synthesized for a given number of plates. However, if all clusters are required to be represented and robotic automation is available, Method 2 will guarantee total coverage. Method 3 will cover slightly more clusters than that obtained by Method 1, but at a price of using more reagents and synthesizing more compounds.

### EXAMPLE B

To demonstrate the advantages of the present invention, the invention was applied to a specific combinatorial library of 10,368 compounds. The Library was generated by substitution at-three locations on a parent structure, as follows:

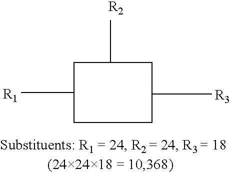

Activity as measured by percent inhibition was determined for all of the compounds except one. Molconn-Z (Edu Soft, Ashland, Va.) and Cerius² (Molecular Simulations Inc, San Diego, Calif.) software were used to generate 249 chemical descriptors for each compound in the library. SAS® (SAS Institute Inc., Cary, N.C.) was used to perform Principal Component Analysis on these descriptors. Fifteen components were selected which accounted for 90% of the variability. This 15-space was clustered by nonhierarchical non-parametric density estimation by various k values (nearest neighbors) using SAS® software (MODECLUS procedure). A k value of 6 was selected by the chemists which gave an acceptable resolution of compounds into clusters (737). The Frequency Distribution Sampling Method as described was used to generate a subset of compounds from the clustered library. The results follow:

There 10,367 compounds that we have percent inhibition data for. Of these there are 8,093 with % inhibition <50%

2,274 with % inhibition>50% or 21%

| | |
|---|---|
| 1,865 | 55% or 18% |
| 1,466 | 60% or 14% |
| 1,139 | 65% or 11% |
| 860 | 70% or 8% |
| 628 | 75% or 6% |
| 408 | 80% or 4% |
| 241 | 85% or 2% |
| 117 | 90% or 1% |
| 44 | 95% or .4% |

There 542 clusters out of the 737 total clusters that contain at least 1 active compound as defined by a percent inhibition greater than 50%. Of these, there are 131 clusters of which only have one of the members that is active. There are 92 clusters of which two are active etc.

| Number | | | | |
|---|---|---|---|---|
| Active | Cluster | | Cumulative | Cumulative |
| Cmpd’s | Frequency | Percent | Frequency | Percent |
| 1 | 131 | 24.2 | 131 | 24.2 |
| 2 | 92 | 17.0 | 223 | 41.1 |
| 3 | 80 | 14.8 | 303 | 55.9 |
| 4 | 54 | 10.0 | 357 | 65.9 |
| 5 | 49 | 9.0 | 406 | 74.9 |
| 6 | 31 | 5.7 | 437 | 80.6 |
| 7 | 26 | 4.8 | 463 | 85.4 |
| 8 | 19 | 3.5 | 482 | 88.9 |
| 9 | 13 | 2.4 | 495 | 91.3 |
| 10 | 14 | 2.6 | 509 | 93.9 |
| 11 | 8 | 1.5 | 517 | 95.4 |
| 12 | 6 | 1.1 | 523 | 96.5 |
| 13 | 2 | 0.4 | 525 | 96.9 |
| 14 | 4 | 0.7 | 529 | 97.6 |
| 15 | 4 | 0.7 | 533 | 98.3 |
| 16 | 2 | 0.4 | 535 | 98.7 |
| 18 | 1 | 0.2 | 536 | 98.9 |
| 19 | 2 | 0.4 | 538 | 99.3 |
| 20 | 1 | 0.2 | 539 | 99.4 |
| 21 | 1 | 0.2 | 540 | 99.6 |
| 24 | 1 | 0.2 | 541 | 99.8 |
| 26 | 1 | 0.2 | 542 | 100.0 |

The distribution of active compounds will vary depending on the clustering method selected, the variables used to describe the compounds in the library, and the nature of the biological activity (e.g. structural specificity). For the above method approximately 50% of the active compounds are distributed in clusters which include 5 or more active compounds.

### Method Statistics

Since there are three substituent locations, this is a 6 (3!=3×2×1) dimensional problem. there are six possible combinations of the three R ranked groups for selection of the 737 cluster representatives: {(1,2,3), (1,3,2), (3,2,1), (3,1,2), (2,1,3), (2,3,1)}. If there is no preferential R group to construct the 10×8 well plates, then there are three possible ways to construct the plates (by R₁, by R₂, or by R₃. This results in eighteen (3 R groups×6 dimensions) possible solutions of dot density reduction. Of this solution space there are two solutions that would require thirty-three plates to cover all of the 737 clusters by robotic automation. The worst solution required fifty plates. One of the two best solutions is:

TABLE VIII
Dimension (1 3 2) by R₁, (Plates are made with r₁, constant for a plate)

| | | | | Cu- | |
|---|---|---|---|---|---|
| | | | Fre- | mu- | |
| | R₁ | | quency | lative | |
| OBS | Structure | Plate | Count | Total | |
| 1 | PhEt | 1 | 80 | 80 | ←1st plate covers 80 clusters |
| 2 | 4MoPh | 1 | 76 | 156 | ←2nd plate covers 76 unique |
| 3 | 2ClPhMe | 1 | 70 | 226 | clusters |
| 4 | PhEt | 2 | 70 | 296 | |
| 5 | 4MoPh | 2 | 58 | 354 | |
| 6 | PhEt | 8 | 52 | 406 | |
| 7 | 2ClPhMe | 2 | 49 | 455 | |
| 8 | 4MoPh | 8 | 31 | 486 | |
| 9 | EtAc | 1 | 30 | 516 | |
| 10 | 2FPh | 1 | 28 | 544 | |
| 11 | PhEt | 9 | 28 | 572 | |
| 12 | 4iPrPh | 1 | 23 | 595 | |
| 13 | 24MoPh | 1 | 17 | 612 | |
| 14 | 2ClPhMe | 8 | 14 | 626 | |
| 15 | 3BrPh | 1 | 14 | 640 | |
| 16 | 3MoPhEt | 1 | 14 | 654 | |
| 17 | 3TMPh | 1 | 13 | 667 | |
| 18 | 2Naphth | 1 | 11 | 678 | |
| 19 | cHex | 1 | 10 | 688 | ←after nineteen plates, the |
| 20 | EtAc | 8 | 7 | 695 | rest of the compounds could |
| 21 | 3GlPr | 1 | 5 | 700 | be made separately, thereby |
| 22 | 4MoPh | 9 | 5 | 705 | decreasing the number of |
| 23 | 2FPh | 8 | 4 | 709 | additional plates. |
| 24 | 2NOPh | 1 | 4 | 713 | |
| 25 | 3BrPh | 2 | 4 | 717 | |
| 26 | 2GlPhMe | 9 | 3 | 720 | |
| 27 | 2MeCOPh | 1 | 3 | 723 | |
| 28 | 3MoPhEt | 2 | 3 | 726 | |
| 29 | 4iPrPh | 8 | 3 | 729 | |
| 30 | EtAc | 2 | 3 | 732 | |
| 31 | Ph | 1 | 3 | 735 | |
| 32 | 24MoPh | 2 | 1 | 736 | |
| 33 | EtAc | 9 | 1 | 737 | |

Of the 737 cluster representative compounds which were specifically selected for this solution there were:

161 with % inhibition>50% or 22% (161/737)

| | |
|---|---|
| 130 | 55% or 18% |
| 108 | 60% or 15% |
| 81 | 65% or 11% |
| 60 | 70% or 8% |
| 46 | 75% or 6% |
| 32 | 80% or 4% |
| 21 | 85% or 3% |
| 9 | 90% or 1% |

Notice the percent actives for the sample selected are in the same proportion as for the entire population.

If thirty-three (8×10 well) plates were generated, 2,640 compounds would be synthesized. If only the R₁, R₂, and R₃ components (reagents) necessary to produced the representative cluster compounds were used then only 1,338 compounds would need to be synthesized (about 50% of the previous total). For example, on the 31st plate 3 additional clusters are covered. To make these 3 compounds a 2×2 matrix of reagents are required. This however will result in 4 compounds generated of which one compound will provide extra representation of some cluster. An 8×10 matrix of reagents would result in 77 extra compounds. Plate 2 requires a 8×10 matrix of reagents however there are only 76 unique clusters covered resulting in 4 duplicated clusters represented. Thus over the 33 plates 1,338 compounds would be synthesized to cover the 737 clusters.

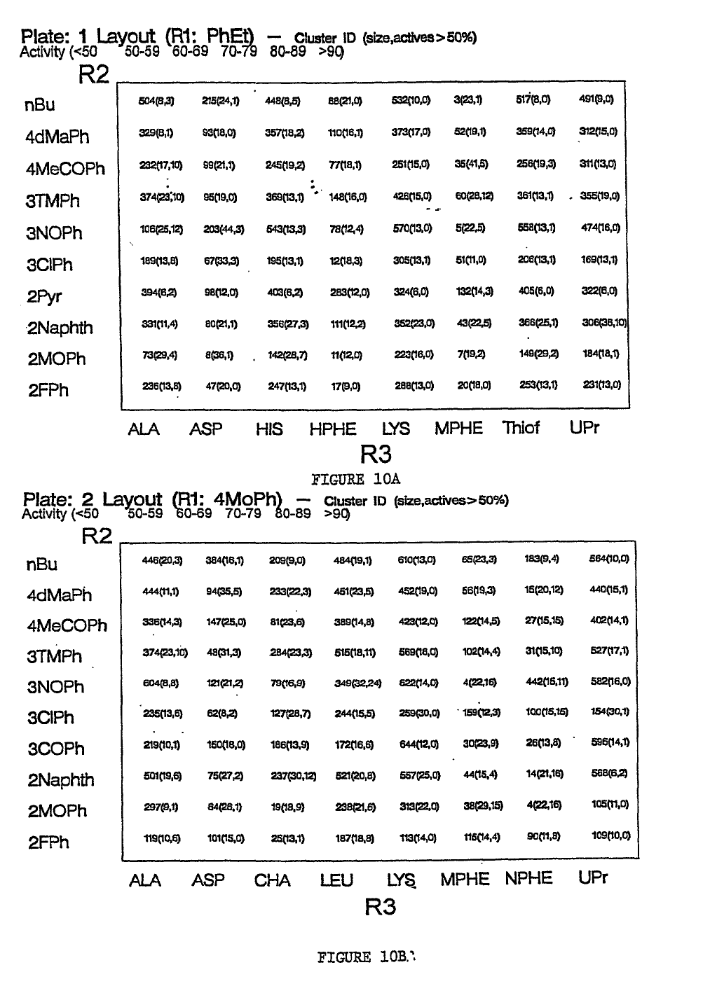

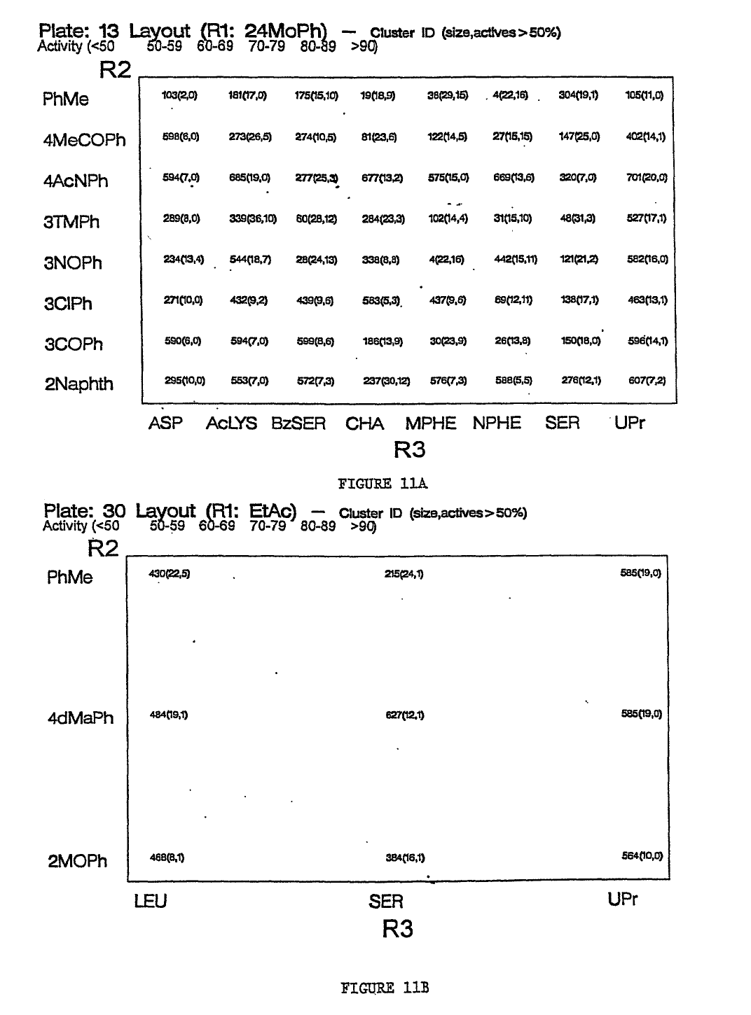

On the 33rd plate, 2 reagents (1×1 matrix) are required to produce 1 additional cluster covered. This one compound might be able to be included with compounds from other plates which also have limited cluster coverage. For example, in this case plate 30 and plate 33 could be combined since the R₁ group (EtAc) is the same for both plates. Although this would eliminate an extra plate, this would also generate 3 extra replicate compounds (see example plate 30 description below, FIG. 11B). Since compound synthesis is performed by robotic technology, this could perhaps be seen as a good tradeoff. (one less plate, three more compounds).

Of the 1,338 compounds generated 737 clusters will be covered and 601 compounds will provide duplicate representation of the clusters. Of these 1,338 compounds 26% were active (>50% inhibition). This sample has an increase of 5% over the actives for the population (21% vs 26%).

Of the 1,338 compounds which were specifically selected for this solution there were:

| | % Sample Activies | % Population Activies |
|---|---|---|
| 348 with % inhibition> | | 21% |
| 50% or 26% (348 1,338) | | |
| 291 | 55% or 22% | 18% |
| 241 | 60% or 18% | 14% |
| 182 | 65% or 14% | 11% |
| 141 | 70% or 10% | 8% |
| 106 | 75% or 8% | 6% |
| 76 | 80% or 6% | 4% |
| 52 | 85% or 4% | 2% |
| 26 | 90% or 2% | 1% |
| 11 | 95% or 0.8% | 0.4% |

At each level the percentage of active compounds in the sample was higher than the overall active percentage in the population and at the most potent levels (>80%) the percentage was approximately double. If the clusters identified as having an active compound were further investigated, there would have been 1,481 active compounds discovered compared with 2,274 for the entire library (or 65%). This leaves 793 compounds that would not have been found. In a small library such as this, it is not a problem to screen the entire library. If the library were very large though, screening the entire library would be very time consuming, expensive or prohibitive. In this example, an initial sample size that was 13% of the original size would lead to 65% of all of the actives. Thus, by using the method of the present invention, 65% of all active compounds could have been determined by sythesizing a selected subset of 1,338 compounds. Accordingly, only 13% of the 10,368 compound library need to have been synthesized, which is a substantial saving in both time and materials, leading to substantial cost savings.

### Examples Plate Configurations

Referring to the drawings, Plate 1 is shown in FIG. 10A In well 1 (first row, first column) a compound was selected from cluster 504. There were 8 compounds in that cluster of which 3 were active. The compound selected from that cluster was not active. This cluster did not have any other duplicate representatives and the 3 active compounds might not have been discovered due to this situation. In well 54 (row 7, column 6) a compound from cluster 132 was selected. There were 14 compounds in that cluster and 3 were active. One of those actives was discovered.

Sometimes patterns can be found within the plate configuration. In column 3 for example there seems to be higher activity associated with the HIS group at location R₃.

Referring to the drawings, Plate 2 is shown in FIG. 10B.

Again there seems to be higher activities corresponding to the R₃ location having a LEU (column 4) or NPHE group (column 7). Note on this plate well 54 (row 7, column 6) which represented cluster 30 had 23 compounds of which 9 were active. The compound selected here though was not active. However, on plate 13 an active compound was selected from this cluster as a cluster duplicate.

Referring to the drawings, Plate 13 is shown in FIG. 11A.

Cluster 30 is selected again (row 7 column 5). However the compound selected this time is active in the 70-80% range.

Referring to the drawings, Plate 30 is shown in FIG. 11B.

As noted above it is possible to consolidate information from plates with low cluster coverage. In the example described it was suggested that the information contained for plates 30 and 33 could be combined in a single plate. Note that the r₁ group required for plates 30 and 33 is EtAc. The reagents for the plates are different as would be expected from the algorithm which generates the plate configuration which optimizes cluster coverage with fewest compounds synthesized. Plate 30 has a 3×3 reagent matrix. Plate 33 has a 1×1 matrix. They could be combined to form a 4×4 matrix resulting in 16 compounds synthesized which is greater than the sum of compounds produced by using the two plates (9+1).

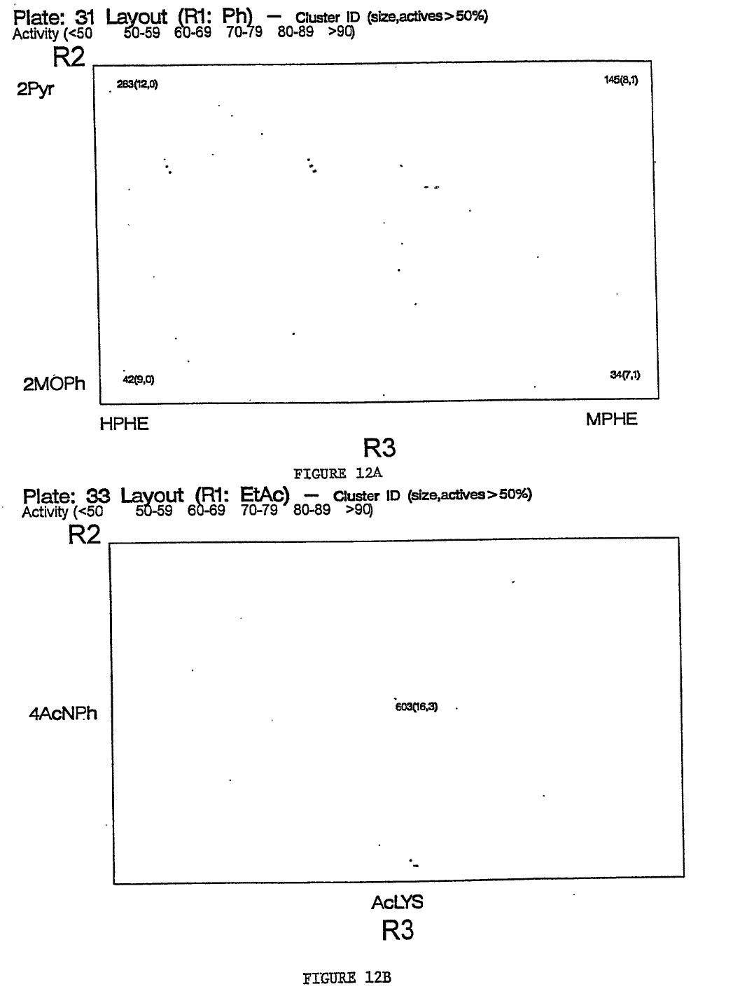

Referring to the drawings, Plate 31 is shown in FIG. 12A.

As described above clusters 145, 42, and 34 are unique representatives. Cluster 283 is represented in an earlier plate. In this case the extra representation was not an advantage since there are no active compounds in cluster 283.

Referring to the drawings, Plate 33 is shown in FIG. 12B.

Plate 33 was necessary to generate a compound from the only cluster remaining that was not represented. In this case cluster 603 had 3 of 16 compounds active though none were chosen.

What is claimed is:

1. In a method for synthesizing compounds from a large chemical library, the improvement which comprises partitioning the library into diversity spaces, analysing substituent frequency of substituents used to prepare chemical compounds in each diversity space and using the resulting analysis to select chemical compounds for synthesis.
